(19) United States
(12) Patent Application Publication
(10) Pub. No.: US 2007/0010459 A1
(43) Pub. Date: Jan. 11, 2007

(54) Application of asiatic acid and its derivatives to treat pulmonary fibrosis

(76) Inventors: Ying Liu, Shanghai (CN); Xingchu Gu, Shanghai (CN); Quanhai Liu, Shanghai (CN); Longhai Shen, Shanghai (CN); Jingzhi Dai, Shanghai (CN); Minyu Liu, Shanghai (CN)

Correspondence Address: ROSENBAUM & ASSOCIATES, 650 DUNDEE ROAD, SUITE 380, NORTHBROOK, IL, 60062-2757, US

(21) Appl. No.: 11/333,030

(22) Filed: Jan. 17, 2006

(30) Foreign Application Priority Data
Jan. 14, 2005 (CN) CN200510023361.9

(51) Int. Cl. A61K 31/704; A61K 31/20

(52) U.S. Cl. 514/26; 514/559

(57) ABSTRACT

Compositions comprising asiatic acid and its derivatives and asiaticoside and the methods of use there of to treat pulmonary fibrosis are provided.

### CROSS-REFERENCE TO RELATED APPLICATIONS

This application claims priority to the Chinese patent application No. 200510023361.9, filed Jan. 14, 2005.

### BACKGROUND OF THE INVENTION

This invention relates generally to the area of natural herbal medicine, specifically the application of asiatic acid in treating pulmonary fibrosis.

The crude extract of Centella asiatica has been used to promote wound healing and inhibit excessive scar proliferation in China, India and other countries.

Centella asiatica, also known as gotu kola, Indian Pennywort and mandookaparni, is a perennial plant native to China, India and other tropical countries and has been used for centuries as a medicinal herb and was referred to in the Indian Ayurvedic medicine some 5000-3,000 years ago, in the ancient traditional Chinese Shennong Herbal some 2,000 years ago, as well as in the French pharmacopoeia since 1884 AD. It is listed in the historic Susruta Samhita, an ancient Indian medical text. In China, gotu kola is one of the reported miracle elixirs of life.

In the nineteenth century, gotu kola and its extracts were incorporated into the Indian pharmacopeia, wherein in addition to being recommended for wound healing, it was recommended in the treatment of skin conditions such as leprosy, lupus, varicose ulcers, eczema, and psoriasis. It was also used to treat diarrhea, fever, amenorrhea, and diseases of the female genitourinary tract. (See http://www.holistic-online.com/Herbal-Med/_Herbs/h18.htm and Poizot. A., Dumez. D, C. R. Acad. Sci [D] 286, 1978). Additional traditional uses of Centella asiatica include heart disease, high blood pressure, rheumatism, fevers, nervous disorders, bronchitis, asthma and syphylis. (Duke, J. A. CRC Handbook of medicinal herbs. Boca Raton, Fla.: CRC Press, 1985, 110-111).

The active compounds of Centella asiatica were first isolated in the early 1940's. (J. E. Bontems, Bull. Sci. Pharmacol., (1941) 49, 186-96). The active ingredients of Centella asiatica were determined to be triterpenoids (also called saponins) the constituents of which include asiatic acid, a triterpene (I), asiaticoside (glycoside of asiatic acid, II), brahmoside, brahminoside, madecassoside, and madecassic acid. Other ingredients include thiamine, riboflavin, pyridoxine, vitamin K, asparate, glutamate, serine, threonine, alanine, lysine, histidine, magnesium, calcium and sodium. (http://www.himalayahealthcare.com/aboutayurveda/cahc.htm#centella).

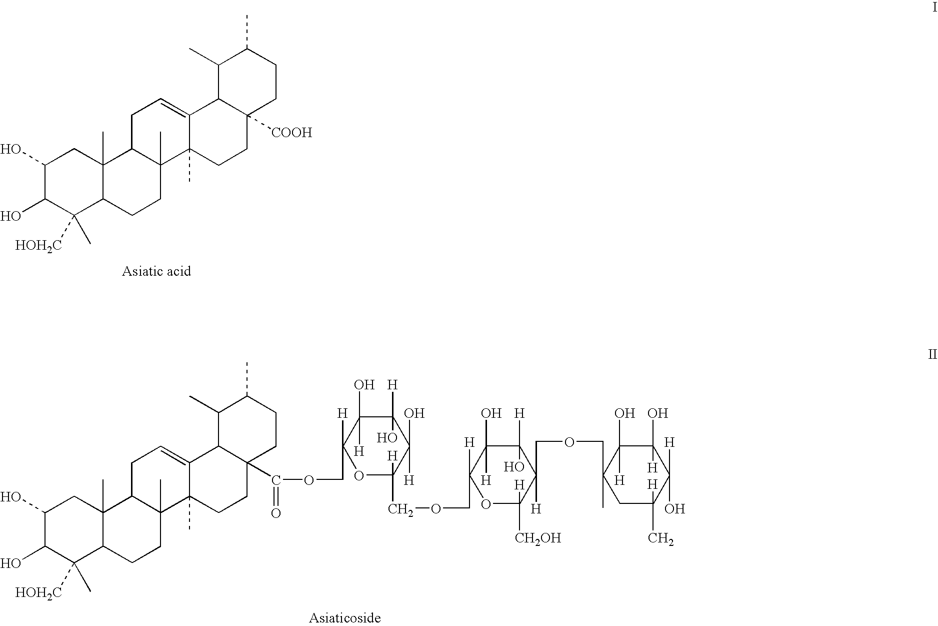

It has been reported that the titrated extract of Centella asiatica is acknowledged as a medicinal effector and commercially available under the tradename of “Madecassol”. Its medicinal effect is to restore a damaged tissue to a nearly original state by modifying the fibrosis progress with protection of the cells in the tissue. The components of Centella asiatica are useful in the curing of surgical wounds (Korean Pat. Publication Nos. 87-1458, 87-1573 and 91-2518). Topical application of extracts purified from Centella asiatica have been shown to aid in wound healing, burns, chronic venous insufficiency and the treatment and prevention of enlarged scar tissue (keloids). (Kartnig T., In Herbs, Spices, and Medicinal Plants: Recent Advances in Botany, Horticulture, and Pharmacology, vol. 3, ed L. E. Craker, J. E. Simon. Phoenix, Ariz.: Oryx Press, 1986, 145-73; Mahajani, S. S., et al., Can J Physiol Pharmacol 1994; 72 (suppl 1):180; Pointel, J. P., et al., Angiology 1986; 37(5):420-21. Bosse, J. P., et al., Ann Plastic Surg 1979; 3:13-21). Isolated extracts may promote wound healing through the activation of malpighean cells and the induction of keratinization. (May, A. Eur. J. Pharmacol., (1968) 4(3), 331-9).

Topical and cosmetic compositions containing Centella asiatica are known for healing skin scars, ulcers and wounds. See, for example, French patent No. 1433383, French patent No. 4209M, French patent Application No. 2594690, French patent Application No. 2696932, UK patent Application No. GB 2309695, International Application No. WO 97/39734, International Application No. WO 98/23574, International Application No. WO 98/23574, International Application No. WO 01/19365, International Application No. WO 97/39734, U.S. Pat. No. 5,834,437, U.S. Pat. No. 6,891,063, U.S. Pat. No. 5,807,555, and U.S. Pat. No. 6,159,494.

Asiatic acid is reported to be the active ingredient of Medecassol. (Rush W R, et al., Eur J Drug Metab Pharmacokinet., (1993) 18 (4):323-6). Asiatic acid derivatives have been further shown to independently function as wound healing agents and protective agents against beta-amyloid induced neurotoxicity. (U.S. Pat. No. 5,834,437; Mook-Jung, I., et al., J. Neurosci. Res., (1999) 58 (3):417-25). Asiatic acid and its derivatives are also known for treating dementia and cognitive disorders. See, for example, International Application No. WO 98/23278, and U.S. Pat. No. 6,169,112.

Asiatic acid and its derivatives are also known for liver function protection, treating hepatotoxicity and proliferating diseases like cancer. See, for example, U.S. Pat. No. 6,417,349, U.S. Patent Application No. 2004/0097463, U.S. Pat. No. 6,274,559, International Application No. 98/23575, and International Application No. 98/37899.

Asiatic acid and asiaticoside form part of various pharmaceutical compositions and mixtures. See, for example, International Application No. WO 2004/0626798, International Application No. WO 01/19158, International Application No. WO 01/19381, U.S. Pat. No. 5,529,769, U.S. Pat. No. 4,707,360, and U.S. Pat. No. 6,756,065.

Various derivatives of asiatic acid and asiaticoside are known. See, for example, U.S. Pat. No. 3,365,442, and U.S. Pat. No. 3,366,669. U.S. Pat. Application No. 20040097463 discloses asiatic acid analogs and their use for the treatment of diseases including cancer.

It is also known that asiatic acid is insoluble in water in general. Madecassol, the titrated extract of Centella asiatica, containing asiaticoside, asiatic acid and madecassic acid in 4:3:3 ratio, is insoluble. A mixture of asiaticoside and madecassoside, in 4:6 or 6:4 ratio, is soluble in water, alcohol, and vegetable oil. Asiatic acid and madecassic acid could be removed from the Centelle asiatica extract by repeated washings with aqueous alkaline solution. See, for example, U.S. Pat. No. 6,417,349.

In further study, the inventors of this application found out that asiaticoside not only inhibits hyperplasy of fibric scars, but also treats pulmonary fibrosis.

Pulmonary fibrosis is the terminal pathological ailment in lung tissues caused by various factors. These factors could be physical, e.g. pneumonia resulting from radiation therapy, silicosis, phosgenismus, or infectious, e.g. chronic bronchitis, TB or schistosome, or vascular, e.g. rheumatic arteritis or primary pulmonary sclerosis, or genetics, e.g. idiopathic pulmonary fibrosis and scleroderma. Once the fibrotic course starts, pulmonary functions are broadly affected and symptoms like dyspnea follow. Once fibrosis is formed, it is believed in modern medicine, this pathological change cannot be reversed. This ailment would seriously impact patients' well being and results in loss of physical working capability and ultimately develop into pneumocardial disease and failure in respiratory system that could cause the death of patients.

The multiple factors that cause pulmonary fibrosis are very common. Therefore addressing the fundamental cause of this aliment would be the ideal approach to contain this disease. Owing to the difficulty totally eliminate all the subjective and objective natures factors causing pulmonary fibrosis, the study on how to inhibit the onset of pulmonary fibrosis becomes a very pressing issue.

Modern biomedical research has invested significant amount of resources in the research of the metabolism of collagen and genesis and development of fibroblast, significant progress have been made in understanding of various components affecting collagen metabolism and cellular factors impacting fibrosis process, e.g. TGF-β, TNF-α, PDGF, IL-1 IL-6, FGF and EGF. So far the most promising molecule seems to be TGF-β, as it significantly increases the content of extrecellular matrix, of fibroblasts, such as Type I and III collagen and firbronectin, which can inhibit the synthesis of collagenase and block the degradation of collagen. TGF-β monoclonal antibody has demonstrated considerable efficacies in treating experimental skin injury and acute glomerular nephritis and post PTCA injury to arterials. Study on small molecules targeting TGF-β receptor is also underway. Screening of antagonists to other cellular factors and endothelins is also a hot topic. There are multiple studies on inhibitors of collagen biosynthesis pathway targeting two key enzymes in the posttranslational modifications of collagen, i.e. praline-4-hydroylase and lysyloxidase. Since matrix metalloproteinase, e.g. collagenase can degrade collagen, therefore agonists to these enzymes are also the focus of the research on fibrosis therapeutic.

According to American Lung Association, several causes of pulmonary fibrosis are known such as Occupational and environmental exposures, sarcoidosis, drugs, radiation, connective tissue or collagen diseases, and genetic/familial. Occupational and environmental exposures due to jobs—particularly those that involve mining or that expose workers to asbestos or metal dusts—can cause pulmonary fibrosis. Workers doing these kinds of jobs may inhale small particles (like silica dusts or asbestos fibers) that can damage the lungs, especially the small airways and air sacs, and cause scarring (fibrosis). Agricultural workers also can be affected. Some organic substances, such as moldy hay, cause an allergic reaction in the lung. This reaction is called Farmer's Lung and can cause pulmonary fibrosis. Other fumes found on farms are directly toxic to the lungs. Sarcoidosis, a disease characterized by the formation of granulomas (areas of inflammatory cells), can attack any area of the body but most frequently affects the lungs. Certain medicines may have the undersirable side effect of causing pulmonary fibrosis. Connective tissue or collagen diseases such as rheumatoid arthritis and systemic sclerosis also cause pulmonary fibrosis.

The treatment options for idiopathic pulmonary fibrosis are very limited. There is no evidence that any medications can help this condition, since scarring is permanent once it has developed. Lung transplantation is the only therapeutic option available. At times, this diagnosis can be difficult to make even with tissue biopsy reviewed by pathologists with specific experience in this field. Research trials using different drugs that may reduce fibrous scarring are ongoing. Since some types of lung fibrosis can respond to corticosteroids (such as Prednisone) and/or other medications that suppress the body's immune system, these types of drugs are sometimes prescribed in an attempt to decrease the processes that lead to fibrosis.

The immune system is felt to play a central role in the development of many forms of pulmonary fibrosis. The goal of treatment with immune suppressive agents such as corticosteroids is to decrease lung inflammation and subsequent scarring. Responses to treatment are variable. Once scarring has developed, it is permanent.

The toxicity and side effects of treatments can be serious. Further, only a minority of patients respond to corticosteroids alone, so other immune-suppressing medications are used in addition to corticosteroids. These include gamma-interferon, cyclophosphamide, azathioprine, methotrexate, penicillamine, and cyclosporine. The anti-inflammatory medication colchicine has also been used with limited success.

Pulmonary fibrosis can cause decreased oxygen levels in the blood. A decrease in blood oxygen level (hypoxia) can lead to elevated pressure in the pulmonary artery (the vessel that carries blood from the heart to the lungs to receive oxygen), a condition known as pulmonary hypertension, which can in turn lead to failure of the right ventricle of the heart. Therefore, patients with pulmonary fibrosis are frequently treated with supplemental oxygen to prevent pulmonary hypertension.

There is also evidence that patients suffering from pulmonary fibrosis may be at increased risk for blood clots that travel to the lung (pulmonary emboli), and therefore anticoagulation (blood thinning) therapy may be indicated.

Those currently used drugs in clinics, such as Colchicine, γ-Interferon, prostaglandin-E analogs, e.g. Malotilate and ursodeoxycholic acid, have unclear mechanisms and less definitive efficacies. Hormone is used to treat idiopathic pulmonary fibrosis patients and effective in some cases. New drugs undergoing clinical development, such as pirfenidone and relaxin are gaining more attention. In some cases like scleroderma and subsequent fibrosis, the efficacy is verified. However, there are recent setbacks in clinical study of these drugs to support their efficacy.

Chinese herbal medicine sometimes provide a unique resource to treat stubborn diseases and the outcome could be very encouraging. So far there is no report on any herbal preparation to treat pulmonary fibrosis.

In the course of study, the inventors discovered asiaticoside (as shown in the Chinese patent application No. 031116105.7, which is incorporated herein in its entirety) and asaitic acid effect on the formation of fibrin in fibroblast culture in vivo.

Asiatic acid can be formed with asiaticoside desurgarizing its polysaccharide moiety. Therefore, the inventors added asiatic acid into the culture medium of fibroblast and found asiatic acid exerts some roles in fibrin formation. Therefore the inventors conclude when asiaticoside is administrated internally, the active form existing in vivo would be asiatic acid.

The inventors have demonstrated the effects of asiaticoside in treating pulmonary fibrosis, it is therefore plausible to use asiatic acid directly as active ingredient in drug composition in clinical application to treat pulmonary fibrosis.

### SUMMARY OF THE INVENTION

One aspect of the invention is the use of a composition for the treatment of fibrosis comprising administering to a subject in need of such treatment an amount of a composition comprising asiatic acid and its derivatives. In another embodiment, the invention further comprises administration to the subject of another antifibrosis compound. In a further embodiment, the additional antifibrosis compound is asiaticoside. In another embodiment, the invention is a use of a composition for inhibiting lung fibrosis comprising asiatic acid and asiaticoside.

Another embodiment of the invention is a pharmaceutical preparation comprising an amount of asiatic acid and its derivatives effective to treat fibrosis, and a pharmaceutically acceptable carrier. Yet another embodiment of the invention is a pharmaceutical preparation comprising an amount of an antifibrosis compound such as asiaticoside effective to treat fibrosis, and a pharmaceutically acceptable carrier.

### DETAILED DESCRIPTION OF THE INVENTION

As used herein, the phrase “asiatic acid and its derivatives” encompasses naturally occurring and synthetic asiatic acid and its derivatives such as asiticoside and also includes the products obtained from the Centella asiatica, including, but not limited to, brahmoside, brahminoside, madecassoside, and madecassic acid.

The technical problem that is addressed in this invention is to use animal model to assess the effects of asiatic acid and its derivatives in treating pulmonary fibrosis.

This invention provides a method of using asiatic acid and its derivatives to treat pulmonary fibrosis.

One aspect of the invention is the use of a composition for the treatment of fibrosis comprising administering to a subject in need of such treatment an amount of a composition comprising asiatic acid and its derivatives. In another embodiment, the invention further comprises administration to the subject of another antifibrosis compound. In a further embodiment, the additional antifibrosis compound is asiaticoside. In another embodiment, the invention is a use of a composition for inhibiting lung fibrosis comprising asiatic acid and asiaticoside.

Another embodiment of the invention is a pharmaceutical preparation comprising an amount of asiatic acid and its derivatives effective to treat fibrosis, and a pharmaceutically acceptable carrier. Yet another embodiment of the invention is a pharmaceutical preparation comprising an amount of an antifibrosis compound such as asiaticoside effective to treat fibrosis, and a pharmaceutically acceptable carrier.

Materials and Methods

Rat is used as experimental animal and commercially available asiatic acid and asiticoside to conduct the described experiments herein.

This invention illustrates the effect of asiatic acid and its derivatives such as asiaticoside on treating pulmonary fibrosis with following experiments.

The pulmonary fibrosis animal model was induced by bleomycin in rat according to the methods described by Lazo J S, Hoyt D G, Sebti S M, Pitt B R. in Pharmacol Ther. 1990: 47:347-58. In this article, praquat, bleomycin and amiodrone are three routinely used chemical agent to induce lung fibrosis is rodents, e.g. mice, rat and Chinese hamsters as well as primates, e.g. baboon and cynomolgous monkey etc. there are also reports describing using rabbit or dog as model animal for such study. Different groups were able to generate lung fibrosis in animal models through different administration routes. For example, 3 various pulmonary fibrosis animal models using Co⁶⁰ to radiate rat, bleomycin or CCl₄ injection into abdominal cavity of mice or rat respectively were generated. While the extent of pathological change is comparable, the time that it took to obtain pulmonary fibrosis model varies between 90, 53 and 35 days respectively. The pathology analysis indicates the occurrence of acute injury of lung and pulmonary fibrosis and therefore such model is suitable for pharmacology studies. Specifically, diffusive stromal lung fibrosis model induced by tracheal bleomycin dripping is the most commonly used animal model. The advantage of this method is that the model can be obtained with once bleomycin dripping without repeated venous or abdominal injections, therefore limiting the influences to other organs caused by systematic administrations. It also lowers the experimental cost. The development of pulmonary fibrosis from alveolitis can be observed in 4 weeks, which is very similar to the pulmonary fibrosis pathological development in human being.

The experiment results indicate that asiatic acid as the active ingredient and form of asiaticoside possesses the activity to relieve pulmonary fibrosis. This invention reveals that direct administration of asiatic acid can relieve lung fibrosis in rats receiving bleomycin induction. The experiments verify the therapeutic efficacy of asiatic acid in treating pulmonary fibrosis.

This invention provides an application of asiatic acid in treating lung fibrosis. Therefore, asiatic acid can be used as an active ingredient to prepare drug composition to treat lung fibrosis. Since asiatic acid monomer can be extracted from Centalla asiatica directly at low cost and high yield, there is a promising perspective to develop asiatic acid as a therapeutic to treat lung fibrosis.

### EXAMPLES

1. Animal: SD male rats with weight raging between 200-250 g, purchased from SLAC.

2. Materials:

Asiatic acid manufactured by Shanghai Institute of Pharmaceutical Industry.

Dexamethasone manufactured by Shanghai Xinyi Pharma, Lot. No. 020202, specification; 5 mg/Ap.

Bleomycin manufactured by Tianjin Taihe Pharmaceutical Co. Ltd., Lot No. 010001, specification: 8 mg/Ap.

3. Dosage: Asiatic acid, 3, 9, 27/kg, po for 28 days.

Experimental Methods

Male SD rats were lay down flat on operating table and anesthetize with 3% Secobarbital. The trachea was exposed after sterilization of cervical part with alcohol. The needle was then inserted into tracheal cartilage interspace. Bleomycin at 5 mg/kg was dipped in. Afterwards, the rats were reared to evenly distribute bleomycin in the lung. The trachea was then sutured. After the rats regained consciousness and were randomly assigned into individual experimental groups, namely asiatic acid treated group with 3, 9.27/kg, po for 28 days, or dexmethasone group with 0.6 mg/kg po 28 days, sham operated and untreated respectively. The regimen was initiated on the second day. Sham-operated and untreated group received saline. After 28 days of treatment, lung weight, hydroyproline content, pathological section and CT scan were measured or conducted.

Lung Weight Coefficient

After 28 days on asiatic acid, rats were sacrificed and dissected. The body and lung weight were measured respectively and used to calculate lung weigh coefficient.

First Experiment Results:

| | | | | Lung | |
|---|---|---|---|---|---|
| Group | n | | Body Weight (g) | Weight (g) | coefficient |
| Sham operated | 10 | x | 395.13 | 1.79 | 0.45 |
| | | SD | 15.07 | 0.08 | |
| Untreated | 6 | X | 281.56 | 2.24 | 0.86 |
| | | SD | 73.13 | 0.26 | |
| Dexmethasone | 7 | X | 269.40 | 1.90 | 0.72 |
| treated | | SD | 53.06 | 0.42 | |
| Asiatic acid - | 9 | X | 334.00 | 2.17 | 0.70 |
| High dose | | SD | 68.20 | 0.36 | |
| Asiatic acid- | 8 | X | 311.78 | 2.02 | 0.66 |
| Medium dose | | SD | 70.30 | 0.36 | |
| Asiatic acid- | 8 | X | 322.22 | 1.97 | 0.66 |
| Low dose | | SD | 64.62 | 0.31 | |

Second Experiment Results:

| | | | | Lung | |
|---|---|---|---|---|---|
| Group | n | | Body Weight (g) | Weight (g) | coefficient |
| Sham operated | 10 | x | 387.13 | 1.68 | 0.43 |
| | | SD | 15.46 | 0.08 | |
| Untreated | 6 | X | 264.18 | 2.35 | 0.89 |
| | | SD | 67.23 | 0.28 | |
| Dexmethasone | 6 | X | 253.77 | 1.62 | 0.69 |
| treated | | SD | 62.35 | 0.38 | |
| Asiatic acid - | 9 | X | 349.08 | 2.26 | 0.65 |
| High dose | | SD | 53.11 | 0.35 | |
| Asiatic acid- | 8 | X | 315.81 | 2.21 | 0.70 |
| Medium dose | | SD | 58.49 | 0.42 | |
| Asiatic acid- | 8 | X | 307.37 | 2.34 | 0.76 |
| Low dose | | SD | 61.29 | 0.51 | |

Third Experiment Results:

| | | | | Lung | |
|---|---|---|---|---|---|
| Group | n | | Body Weight (g) | Weight (g) | coefficient |
| Sham operated | 10 | x | 399.15 | 1.76 | 0.44 |
| | | SD | 63.17 | 0.44 | |
| Untreated | 6 | X | 281.56 | 2.24 | 0.86 |
| | | SD | 73.13 | 0.26 | |
| Dexmethasone | 7 | X | 269.40 | 1.90 | 0.72 |
| treated | | SD | 53.06 | 0.42 | |
| Asiatic acid - | 9 | X | 334.00 | 2.17 | 0.70 |
| High dose | | SD | 68.20 | 0.36 | |
| Asiatic acid- | 8 | X | 311.78 | 2.02 | 0.66 |
| Medium dose | | SD | 70.30 | 0.36 | 0.12 |
| Asiatic acid- | 8 | X | 322.22 | 1.97 | 0.66 |
| Low dose | | SD | 64.62 | 0.31 | 0.27 |

Hydroyproline Content Measurement.

After 28 days on Asiatic acid, hydroxyproline content in lung was measured. The results are as follows:

First Experiment Results:

| | | | HX content |
|---|---|---|---|
| Group | Dose and admin routes | N | (mg/g) X ± SD |
| Sham operated | Saline * 28 | 10 | 2.38 ± 0.48 |
| Untreated | Saline * 28 | 6 | 3.31 ± 0.88 |
| Dexmethasone | 0.6 mg/kg po * 28 | 7 | 2.34 ± 9.54 |
| treated | | | |
| Asiatic acid-high | 27 mg/kg po * 28 | 9 | 2.39 ± 0.44 |
| dose | | | |
| Asiatic acid- | 9 mg/kg po * 28 | 8 | 2.36 ± 0.87 |
| medium dose | | | |
| Asiatic acid-low | 3 mg/kg po * 28 | 8 | 3.43 ± 0.62 |
| dose | | | |

Second Experiment Results:

| | | | HX content |
|---|---|---|---|
| Group | Dose and admin routes | N | (mg/g) X ± SD |
| Sham operated | Saline * 28 | 10 | 2.34 ± 0.45 |
| Untreated | Saline * 28 | 5 | 3.36 ± 0.76 |
| Dexmethasone | 0.6 mg/kg po * 28 | 5 | 2.41 ± 0.45 |
| treated | | | |
| Asiatic acid-high | 27 mg/kg po * 28 | 9 | 2.37 ± 0.41 |
| dose | | | |
| Asiatic acid- | 9 mg/kg po * 28 | 8 | 2.36 ± 0.79 |
| medium dose | | | |
| Asiatic acid-low | 3 mg/kg po * 28 | 7 | 3.41 ± 0.60 |
| dose | | | |

Third Experiment Results:

| | | | HX content |
|---|---|---|---|
| Group | Dose and admin routes | N | (mg/g) X ± SD |
| Sham operated | Saline * 28 | 10 | 2.37 ± 0.45 |
| Untreated | Saline * 28 | 6 | 3.52 ± 0.86 |
| Dexmethasone | 0.6 mg/kg po * 28 | 7 | 2.47 ± 0.45 |
| treated | | | |
| Asiatic acid-high | 27 mg/kg po * 28 | 9 | 2.30 ± 0.46 |
| dose | | | |
| Asiatic acid- | 9 mg/kg po * 28 | 8 | 2.71 ± 0.44 |
| medium dose | | | |
| Asiatic acid-low | 3 mg/kg po * 28 | 8 | 3.42 ± 0.57 |
| dose | | | |

CT Scanning Results:

After 28 days on asiatic acid, lung CT scans were performed to each group of experimental rats. The results from 3 experiments were similar.

| | |
|---|---|
| Sham operated | No apparent pathological changes visible |
| Untreated | Fibrotic streaks in bilateral lungs and honeycomb lung. |
| | Pleural membrane sags. Consolidation in pulmonary |
| | lobules |
| Dexmethasone | Light hyperplasy in lung striation. Light consolidation |
| treated | in bilateral lungs. |
| Asiatic acid-high | Light glass infiltration, part of lobules consolidates. |
| dose | |
| Asiatic acid- | Fibrotic streaks, consolidation in lobules and |
| medium dose | honeycomb lung. |
| Asiatic acid-low | Consolidation in lobules, bronchiectasis, honeycomb |
| dose | lung |

CT results indicate asiatic acid displays certain therapeutic effects on lung fibrosis. In acute phase of pulmonary fibrosis, while dexmethasone treatment significantly relieved the inflammation, asiatic acid didn't display any therapeutic effects. However, in mid to late phase of the disease, both dexmethasone and asiatic acid can significantly relive the extent of fibrosis in lungs. This result implies asiatic acid may achieve this effect through the inhibition of formation of fibers.

Pathology Results:

After 28 days on asiatic acid, the rats were sacrificed and dissected. Both lungs were harvested. The specimen were embedded and fixed then subjected to HE staining. The focus of infection were observed under microscope with ⅓, ⅔ and 3/3 Vf*10 to conduct semi-quantative analysis.

Results: in untreated group, light to medium focal atelectasis and compensatory emphysema were observed. Alveolar wall thickening to certain extent. Visible fibrocyte and fibroblast proliferation and lymphocyte infiltration. Bronchiole light proliferation. Observe thrombus in capillary and consolidation in arteriole.

In asiatic acid treated group with high, medium and low dose respectively, light fibroplasias in alveoli pulmonis diaphragm, light lymphocyte infiltration and compensatory emphysema. Alveolar wall thickening to very light extent. Light proliferation in fibrous tissues. No thrombus in capillary and consolidation in arteriole were seen. However, the observation is not dose dependent.

In dexmethasone treated group for 28 days, the pathology results were similar to those in asiatic acid treated group.

Cell Biology Experiments Results:

Using bFGF (0.5□g/ml), the proliferation of primary culture of mice fibroblast with the presence of asiaticoside or asiatic acid at 25, 12.5, 6.25, 3.125 or 1.56 μg respectively was assessed.

It is found that asiaticoside displays no apparent inhibitory activity, asiatic acid exerts growth inhibitory roles to fibroblast treated with bFGF to certain extent. The IC50=137.05. This result confirms that the active ingredient with therapeutic effects is asiatic acid.

PK Study Results:

SD rats were given asiaticoside through intragastric administration at 100 mg/kg. The blood samples were taken 0.5, 1, 2, 4, 6, 9, 12 and 24 hours after administration respectively. Plasma samples were prepared and analyzed for asiaticoside and asiatic acid concentration by LC/MS or GC/MS respectively. The analysis indicates there is only trace quantity of asiaticoside in the plasma sample and asiatic acid is present at very high concentration. The plasma concentration of asiatic acid peaked after 4-6 hours after administration.

In conjunction of cell biology experiment results, asiatic acid is confirmed as the active form existing in vivo.

Pharmacodynamics Study of Asiaticoside on Treating Rats with Pulmonary Fibrosis Induced by Bleomycin

The rats with lung fibrosis were induced by bleomycin through trachea administration and then treated with asiaticoside. The asiaticoside was administered at 4, 12 and 36 mg/kg respectively second day after bleomycin induction. Dexamethasone was orally administered as positive control with daily dosage of 0.6 mg/kg. The study regimen lasted for 28 days. The lung weight coefficient and hydroyproline (HXP) content were measured on day 2 and 8 after the formation of animal model. CT scan, pathology and serum ECM content measurements were conducted on day 14, 21 and 28 days respectively. These above results indicate asiaticoside is efficacious in preventing and treating lung fibrosis induced by bleomycin to certain extent.

A. Lung Weight Coefficient and HXP Content Comparison:

| | Lung weight | HXP content |
|---|---|---|
| Group | coefficient | (mg/g) |
| Sham operated | 0.46% | 17.66 ± 3.72 |
| Untreated | 0.88% | 26.56 ± 7.04 |
| Dexamethasone treated | 0.72% | 19.32 ± 4.35 |
| Asiaticoside treated - 4 mg/kg | 0.63% | 19.63 ± 3.91 |
| Asiaticoside treated - 12 mg/kg | 0.66% | 18.95 ± 7.00 |
| Asiaticoside treated - 36 mg/kg | 0.64% | 18.56 ± 3.98 |

B. Collagen Content Comparison

| | | | | Hyaluronic |
|---|---|---|---|---|
| Group | PIIIP | Type IV | Laminin | Acid |
| Sham Operated | 19.03 ± 1.84 | 7.65 ± 4.40 | 41.57 ± 21.21 | 120.89 ± 25.87 |
| Untreated | 45.91 ± 26.88 | 17.55 ± 5.13 | 67.92 ± 13.65 | 327.82 ± 174.45 |
| Dexamethasonetreated | 19.59 ± 6.24 | 10.71 ± 4.94 | 48.41 ± 14.12 | 232.97 ± 101.24 |
| Asiaticoside- | 13.87-24.54 | 4.33-10.45 | 43.83-55.75 | 112.66-312.28 |
| treated (3 doses) | | | | |

C. Cytokine Content Measurement

| Group | TNF-α | TNF-□ | ET-1 |
|---|---|---|---|
| Sham operated | 5.45 ± 0.77 | 0.068 ± 0.007 | 281.41 ± 58.26 |
| Untreated | 6.73 ± 0.26 | 0.084 ± 0.022 | 321.00 ± 48.22 |
| Dexamethasone- | 6.50 ± 0.73 | 0.075 ± 0.010 | 257.86 ± 20.81 |
| treated | | | |
| Asiaticoside- | 5.20-6.49 | 0.067-0.077 | 229.62-241.60 |
| treated (3 doses) | | | |

D. CT Scan Findings

In the Control group, no lesions were found in the bilateral lungs. In the Model Group, glass infiltration was found in lungs, with fibrotic streaks and honeycomb lung and pleural membrane sags. Consolidation in the pulmonary lobules was found indicating formation of pulmonary fibrosis.

In the dexamethasone group, ground glass infiltration and fibrotic streaks were found in both lungs. In the asiaticoside group, ground glass infiltration in the lung tissue was found, but was significantly less than in the model group. This observation was not dose dependent.

E. Pathology

In the Control group: normal alveoli pulmonis, alveolar saccules and respiratory bronchiole were found. In the Model group, however, collapsed alveoli pulmonis, fibrocyte proliferation and lymphocyte infiltration were found with focal atelectasis and compensatory emphysema being observed. The extent of pulmonary fibrosis was aggravated by days after the formation of the model.

In the dexamethasone group, a light inflammatory reaction caused by lymphocyte was found with inflammatory cell infiltration seen in the alveoli pulmonis. Light hyperplasy was seen in alveolar septum with fibroblast proliferation. Slight portion of alveoli pulmonis collapsed along with emphysema. In the asiaticoside group, light fibroplasias in alveoli pulmonis diaphragm, light lymphocyte infiltration and compensatory emphysema were found.

Pharmacodynamics Study on Asiaticoside in Treating Radiation Pneumonia Induced by Cobalt-60 Radiation

Radiation pneumonia and subsequent pulmonary fibrosis in rats was induced by Cobalt-60 radiation. The efficacies were assessed by the lung weight coefficient, HXP content, serum ECM measurement, CT scan and pathological examinations. These tests indicate asiaticoside is efficacious in treating pulmonary fibrosis induced by radiation to certain extent.

A. Lung Weight Coefficient and HXP Content Comparison:

| | Lung weight | HXP |
|---|---|---|
| Group | coefficient | content (mg/g) |
| Sham operated | 0.34% | 29.81 ± 1.87 |
| Untreated | 0.48% | 34.18 ± 6.24 |
| Dexamethasone treated | 0.42% | 36.42 ± 2.74 |
| Asiaticoside treated - 4 mg/kg | 0.42% | 40.88 ± 9.77 |
| Asiaticoside treated - 12 mg/kg | 0.20% | 32.78 ± 4.37 |
| Asiaticoside treated - 36 mg/kg | 0.34% | 31.38 ± 4.75 |

B. Collagen Content Comparison

| Group | PIIIP | Type IV | Laminin | Hyaluronic Acid |
|---|---|---|---|---|
| Sham Operated | 19.43 ± 18.81 | 9.77 ± 3.20 | 34.33 ± 12.56 | 108.39 ± 17.88 |
| Untreated | 32.35 ± 18.37 | 19.97 ± 11.35 | 55.38 ± 19.67 | 324.68 ± 53.12 |
| Dexamethasone- | 14.51 ± 5.61 | 12.65 ± 2.87 | 47.91 ± 8.04 | 195.12 ± 55.89 |
| treated | | | | |
| Asiaticoside | 21.00 ± 9.50 | 14.74 ± 4.03 | 39.21 ± 5.69 | 259.01 ± 64.39 |
| 4 mg/kg | | | | |
| Asiaticoside | 18.84 ± 10.70 | 11.97 ± 4.09 | 41.11 ± 9.15 | 246.91 ± 33.2 |
| 12 mg/kg | | | | |
| Asiaticoside | 14.27 ± 10.40 | 10.04 ± 5.69 | 48.29 ± 10.67 | 220.64 ± 67.55 |
| 36 mg/kg | | | | |

C. CT Scan Findings

No lesions in bilateral lungs were found in the Control Group, whereas in the Model group, ground glass infiltration was found in the right middle and lower lobes, with atelectasis and inflammatory changes with fibrotic streaks being noted in the right lower lobe.

In the dexamethasone group, mild ground glass infiltration and fibrotic streaks in the right lung with right base atelectasis was found.

In the low dose asiaticoside group mild ground glass infiltration and atelectasis in the right lower lung was found. In the median dose, asiaticoside group mild ground glass infiltration and fibrotic streaks in the right lung was found. In the high dose asiaticoside group the right lung exhibited a few fibrotic streaks.

D. Pathology

No pathologic findings were seen in lungs of the control group.

The following pathologic features were observed in the pulmonary fibrosis models: alveolitis (infiltration of chronic inflammatory cells in the alveolar septa, predominantly lymphocytes); focal septal fibrosis and thickening (moderate proliferation of fibroblasts), mild subpleural alveolar collapse and emphysema. In summary, a rat model for pulmonary fibrosis was successfully established.

After treating with asiaticoside at different dosages, similar but milder pathologic changes were observed in the pulmonary fibrosis rat models: focal lymphocytic infiltration and alveolitis, septal fibrosis with moderate proliferation of fibroblasts, and mild subpleural alveolar collapse or emphysema. Therefore, asiaticoside might have some therapeutic effect on rat pulmonary fibrosis, although not dose-dependent.

Pharmacokinetics

Asiaticoside (triterpenes) was extracted from the herb Centella asiatica with 95% purity. The remaining 5% impurity includes asiatic acid, hydroxyl asiaticoside. asiatic acid is the metabolite of asiaticoside with pharmacodynamic effects.

Three doses of asiaticoside (10, 33, 100 mg/kg) were gavaged to rats (n=6) and canines (n=4). After dosing, blood was sampled up to 120 or 216 hours in rats and canines respectively. Tissue distribution and excretion studies in rats were carried out with the highest dose.

The respective asiaticoside and asiatic acid in the same bio-matrix were detected by LC/MS/MS and GC/MS. Because the secondary peak resembled a plasma concentration-time profile in both rat and canine rather than conventional compartment fitting, statistical moment analysis was used in calculating pharmacokinetic parameters. There was no apparent tissue accumulation of asiaticoside and asiatic acid. In addition to the targeted organ, both asiaticoside and asiatic acid were distributed in some other organs, which could be the sign to expand clinical indications.

The main excretion route was by feces, bile and urine. An enterohepatic circulation could exist.

Toxicity Study Summary

Chronic Toxicity Study in Beagle Dogs

Beagle dogs were given 18, 72 or 240 mg/kg asiaticoside respectively daily for 9 consecutive months along with a control group. There is no apparent toxicity was observed in animal behavior, activity, weight, mental state, excrement and urine. No drug related abnormality was observed in hematology, serum biochemistry, urine routine, systematic autopsy, histology examination, and organ weight coefficient.

Based on 9-month chronic toxicity study in Beagle dogs, the nontoxic dose for asiaticoside can be 240 mg/kg/d, which is 40× of proposed clinical dosage in human.

Acute Toxicity Study in Beagle Dogs

Single administration of asiaticoside to Beagle dogs does not result in any toxicity therefore no acute LD₅₀ could be measured. Therefore the highest dosage was given to the animal. The highest dose given was 10 g/kg and no toxicity was observed in Beagle dogs. Food uptake and activity were normal in the observation period. No macroscopic pathological abnormality was observed in autopsy after the animals were sacrificed. Similar acute toxicity results were observed in mice study as well.

Embryonic and Fetus Toxicity and Teratogenicity Test in SD Rats

SD rats were given 120, 380 or 1200 mg/kg of asiaticoside respectively through intragastric administration for 10 consecutive days from day 6-15 of gestation. The results indicated no embryonic and fetus toxicity was observed. Nor there were any fetus anomalies or obform. At 1.2 g/kg, the incidence rate of enlargement of fetus pelvis renalis increased, yet this was not statistically significant. Therefore at 1.2 g/kg, which is 200× the effective dose (6 mg/kg) in mice, there was no embryonic and fetus toxicity.

General Reproductive Toxicity Study in Rats

SD rats were intragastric administrated with 120, 380 or 1200 mg/kg of asiaticoside respectively. Male and female were given 4 weeks or two weeks of asiaticoside before combining the cages. The male ones were given 5 additional weeks of asiaticoside, while female ones were given the drug till the 15^{th} day of gestation. At 1.2 g/kg, evidence of bleeding around apertura narium was noticed in both male and female animal. Yet no abnormality was observed in weight, mating and pregnancy rate, numbers of corepus luteum, nidation, dead fetus, live fetus. Weight, contour, internal organs and skeleton of fetus were all normal. Therefore asiaticoside has no reproductive toxicity.

While the present invention has been described with reference to its embodiments, one of ordinary skill in the relevant art will understand that the present invention is not intended to be limited by these embodiments, and is instead contemplated to include all embodiments consistent with the spirit and scope of the present invention as defined by the appended claims. The entire disclosures of all references, applications, patents, and publications cited herein are hereby incorporated by reference.

We claim:

1. A method of treatment or inhibition of fibrosis comprising administering to a subject in need of such treatment a therapeutically effective amount of a composition comprising asiatic acid and its derivatives.

2. The method according to claim 1, further comprising administering to the subject another antifibrosis compound.

3. The method according to claim 1, wherein the fibrosis is pulmonary fibrosis.

4. A composition for treating or inhibiting fibrosis comprising a compound selected from the group consisting of asiatic acid and its derivatives and asiaticoside, effective to inhibit the fibrosis.

5. The composition of claim 4, wherein the molecule is asiatic acid.

6. The composition of claim 4, wherein the composition further comprises of an additional anti-fibrosis compound in combination.

7. The composition of claim 6, wherein the compound is asiatic acid and the additional anti-fibrosis compound is asiaticoside.

8. The composition of claim 4, wherein the fibrosis is pulmonary fibrosis.

9. Use of asiatic acid and its derivatives in the preparation of a medicament for the treatment of fibrosis.
